(19) United States
(12) Patent Application Publication
(10) Pub. No.: US 2026/0091010 A1
(43) Pub. Date: Apr. 2, 2026

(54) COMPOSITIONS AND METHODS FOR MANAGING REBOUND OF BODY WEIGHT AND METABOLIC PARAMETERS

(71) Applicant: EFIL BIOSCIENCE INC., Daejeon (KR)

(72) Inventors: Jeong-Hoon KIM, Daejeon (KR); Mu Lim CHOI, Daejeon (KR); Kee-Hong KIM, West Lafayette, IN (US)

(21) Appl. No.: 19/346,447

(22) Filed: Sep. 30, 2025

Related U.S. Application Data
(60) Provisional application No. 63/700,942, filed on Sep. 30, 2024.

(51) Int. Cl. A61K 31/185; A61K 31/167; A61K 31/437; A61K 31/4545; A61K 31/496; A61K 38/26; A61P 3/04; A61P 3/06; A61P 3/10; A61P 21/00

(52) U.S. Cl. CPC A61K 31/185; A61K 31/167; A61K 31/437; A61K 31/4545; A61K 31/496; A61K 38/26; A61P 3/04; A61P 3/06; A61P 3/10; A61P 21/00

(57) ABSTRACT

The present invention provides compositions comprising one or more ACAT inhibitors and one or more GLP-1 receptor agonists (GLP-1 RAs). The combination therapy reduces side effects associated with high-dose GLP-1 RA monotherapy and provides durable management of weight loss by suppressing rebound of body weight, blood glucose, and cholesterol levels after treatment.

### CROSS-REFERENCE TO RELATED APPLICATION

The present application claims priority to U.S. Patent Application No. 63/700,942 filed Sep. 30, 2024, which is incorporated herein by reference.

### TECHNICAL FIELD

The present invention relates to compositions and methods for weight management and metabolic regulation, more particularly to approaches that reduce body weight, preserve lean mass, and suppress or delay rebound of body weight, blood glucose, and cholesterol following treatment.

### BACKGROUND

The hallmark of obesity is fat mass enlargement, resulting from an increase in adipocyte cell size and number. In hypertrophied adipocytes, free cholesterol has an augmented uptake, leading to its accumulation in lipid droplets in proportion to increased triglyceride content. B. R. Krause, A. D. Hartman, Adipose tissue and cholesterol metabolism, J Lipid Res 25 (1984) 97-110; F. T. Doole, T. Kumarage, R. Ashkar, M. F. Brown, Cholesterol Stiffening of Lipid Membranes, J Membr Biol. 255 (2022) 385-405, which are incorporated herein by reference. Acyl-coenzyme A: cholesterol acyltransferase (ACAT) that catalyzes the conversion of free cholesterol to cholesteryl ester (CE) using adenosine triphosphate and coenzyme A plays an important role in cellular cholesterol storage. S. Mukherjee, G. Kunitake, R. B. Alfin-Slater, The esterification of cholesterol with palmitic acid by rat liver homogenates, J Biol Chem 230 (1958) 91-96, which is incorporated herein by reference. Increased ACAT1 expression is shown to be associated with increased adiposity and adipogenesis in vitro. Y. Zhu, C. Y. Chen, J. Li, J. X. Cheng, M. Jang, K. H. Kim, In vitro exploration of ACAT contributions to lipid droplet formation during adipogenesis, J Lipid Res 59 (2018) 820-829. 10.1194/jlr.M081745; Y. Xu, X. Du, N. Turner, A. J. Brown, H. Yang, Enhanced acyl-CoA: cholesterol acyltransferase activity increases cholesterol levels on the lipid droplet surface and impairs adipocyte function, J Biol Chem 294 (2019) 19306-19321, which are incorporated herein by reference. Inhibition of ACAT activity was reported to suppress lipid droplet formation and expansion during adipogenesis in vitro. Y. Zhu, C. Y. Chen, J. Li, J. X. Cheng, M. Jang, K. H. Kim, In vitro exploration of ACAT contributions to lipid droplet formation during adipogenesis, J Lipid Res 59 (2018) 820-829, which is incorporated herein by reference. Additionally, it was recently reported that ACAT inhibitor induced significant weight loss while concurrently suppressing food intake in diet-induced obese (DIO) mice with lower blood levels of markers associated with obesity and insulin resistance. Y. Zhu, S. Q. Kim, Y. Zhang, Q. Liu, K. H. Kim, Pharmacological inhibition of acyl-coenzyme A: cholesterol acyltransferase alleviates obesity and insulin resistance in diet-induced obese mice by regulating food intake, Metabolism 123 (2021) 154861, which is incorporated herein by reference. It was reported that an ACAT inhibitor can lower body weight and food intake. U.S. Pat. No. 11,065,216, which is incorporated herein by reference.

It was reported that glucagon receptor (GCGR), glucagon-like peptide-1 receptor (GLP-1R), and glucose-dependent insulinotropic polypeptide receptor (GIPR) involve in regulation of blood glucose and body weight. Cell 187, Christine M. Kusminski et al, Transforming obesity: The advancement of multi-receptor drugs, Jul. 25, 2024:3829-3853. It was also reported that not only GLP-1R mono-agonists and but also GLP-1R multi-agonists including GLP-1R/GCGR co-agonists, GLP-1R/GIPR co-agonists, and GLP-1R/GIPR/GCGR tri-agonists can be used to treat diabetes and obesity. Cell 187, Christine M. Kusminski et al, Transforming obesity: The advancement of multi-receptor drugs, Jul. 25, 2024:3829-3853. For example, GLP-1R agonists, such as semaglutide and liraglutide, were reported to lower body weight and treat type 2 diabetes. Diabetes Ther. 2020 September; 11 (9): 1965-1982; U.S. Pat. Nos. 8,129,343; and 9,993,430, which are incorporated herein by reference. A series of clinical trials named Semaglutide Treatment Effect in People with obesity (STEP) have successfully shown 14.9%-17.4% weight loss effect by once-weekly sc injection of semaglutide 2.4 mg after 68 weeks. T. A. Wadden, T. S. Bailey, L. K. Billings, M. Davies, J. P. Frias, A. Koroleva, I. Lingvay, P. M. O'Neil, D. M. Rubino, D. Skovgaard, S. O. R. Wallenstein, W. T. Garvey, Effect of Subcutaneous Semaglutide vs Placebo as an Adjunct to Intensive Behavioral Therapy on Body Weight in Adults With Overweight or Obesity: The STEP 3 Randomized Clinical Trial, JAMA 325 (2021) 1403-1413. 10.1001/jama.2021.1831; and D. Rubino, N. Abrahamsson, M. Davies, D. Hesse, F. L. Greenway, C. Jensen, I. Lingvay, O. Mosenzon, J. Rosenstock, M. A. Rubio, G. Rudofsky, S. Tadayon, T. A. Wadden, D. Dicker, Effect of Continued Weekly Subcutaneous Semaglutide vs Placebo on Weight Loss Maintenance in Adults With Overweight or Obesity: The STEP 4 Randomized Clinical Trial, JAMA 325 (2021) 1414-1425. 10.1001/jama.2021.3224, which are incorporated herein by reference. The success of semaglutide in treating obesity, resulting in double-digit percentage of body weight loss, has led to its clinical approval (e.g., Ozempic and Wegovy). Similarly, GLP-1R/GIPR co-agonists such as tirzepatide have also demonstrated significant body weight reduction and have subsequently received clinical approval for their effectiveness in treating obesity. M. J. Nauck, T. Quast, P. Wefers, M. Meier, Tirzepatide Versus Semaglutide Once Weekly in Patients with Type 2 Diabetes, NEJM 385 (2021) 503-515. 10.1056/NEJMoa2107519, which is incorporated herein by reference.”

While these therapies have shown significant weight loss results, they are also associated with side effects such as gastrointestinal disturbances at high doses. Additionally, treatment periods often result in muscle loss alongside fat reduction. Upon discontinuation, a significant weight rebound occurs, often characterized by a disproportionately higher regain of fat compared to muscle. Moreover, elevated blood glucose and cholesterol levels have been observed post-treatment. Therefore, novel therapeutic strategies are required to mitigate these side effects and improve long-term outcomes in managing obesity.

This BACKGROUND section serves to provide context for the present disclosure, offering insight into the challenges addressed by the disclosed invention. The statements made herein are intended to facilitate a clearer understanding and are not to be construed as admissions regarding prior art.

### SUMMARY

An aspect of the present invention provides a method for controlling body weight comprising administering to a subject in need one or more ACAT inhibitors and one or more GLP-1 RAs. In some embodiments, the method may comprise administering to a subject a combination of one or more GLP-1 receptor agonists (GLP-1 RAs) and one or more ACAT inhibitors, wherein the combination therapy achieves at least one of the following: (a) enables the use of a lower dose of GLP-1 RA compared to GLP-1 RA monotherapy; (b) provides a weight reduction effect that is comparable to or greater than that of GLP-1 RA monotherapy; (c) reduces muscle loss during the treatment period compared to GLP-1 RA monotherapy; (d) delays and reduces weight regain after cessation of the treatment compared to GLP-1 RA monotherapy; (c) minimizes the rebound in blood cholesterol levels after cessation of the treatment compared to GLP-1 RA monotherapy; and (f) minimizes the rebound in blood glucose levels after cessation of the treatment compared to GLP-1 RA monotherapy. In addition to the above-described effects, the combination therapy may also result in a reduction of muscle mass loss during the treatment period. Furthermore, following the cessation of the treatment, the combination therapy may promote an increase in muscle mass or muscle ratio, providing a unique advantage over GLP-1 RA monotherapy.

In some certain embodiments, the GLP-1 RA may be selected from the group consisting of lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide. In some certain embodiments, the ACAT inhibitor may be selected from the group consisting of avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B. In some embodiments, the GLP-1 RA may be selected from the group consisting of semaglutide, liraglutide, and tirzepatide, and the ACAT inhibitor may be avasimibe, CI-976, eflucimibe, or a pharmaceutically acceptable salt, derivative, or analog thereof.

In some certain embodiments, the GLP-1 RA and the ACAT inhibitor may be formed in a formulation and the formulation is administered. In some embodiments, the GLP-1 RA and the ACAT inhibitor may be formulated separately and administered simultaneously or sequentially.

In some certain embodiments, the combination therapy of one or more GLP-1 RAs and one or more ACAT inhibitors results in unexpected weight reduction effects even when the ACAT inhibitor is administered at a dose that, when used alone, does not cause significant weight loss. Surprisingly, when this lower dose of ACAT inhibitor is combined with a GLP-1 RA, the combination produces a synergistic effect, leading to a pronounced reduction in body weight that is comparable to or greater than the effect seen with higher doses of ACAT inhibitors or GLP-1 RAs alone.

In some certain embodiments, the combination of one or more GLP-1 RAs and one or more ACAT inhibitors allows for a significant reduction in the dose of GLP-1 RA required to achieve weight reduction. When administered in combination with an ACAT inhibitor, the GLP-1 RA dose can be lowered compared to the dose used in GLP-1 RA monotherapy, while still producing comparable or even enhanced weight loss effects. This reduction in GLP-1 RA dosage minimizes potential side effects associated with higher doses of GLP-1 RAs, such as gastrointestinal disturbances, without compromising the therapeutic efficacy.

Another aspect of the present invention provides a pharmaceutical composition for reducing body weight in a subject, the composition comprising one or more GLP-1 receptor agonists (GLP-1 RAs) and one or more ACAT inhibitors. In some embodiments, the composition may be formulated to achieve at least one of the following: (a) enables the use of a lower dose of GLP-1 RA compared to GLP-1 RA monotherapy; (b) provides a weight reduction effect that is comparable to or greater than that of GLP-1 RA monotherapy; (c) reduces muscle loss during the treatment period compared to GLP-1 RA monotherapy; (d) delays and reduces weight regain after cessation of the treatment compared to GLP-1 RA monotherapy; (c) minimizes the rebound in blood cholesterol levels after cessation of the treatment compared to GLP-1 RA monotherapy; and (f) minimizes the rebound in blood glucose levels after cessation of the treatment compared to GLP-1 RA monotherapy.

In some embodiments, the GLP-1 RA may be selected from the group consisting of lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide. In some embodiments, the ACAT inhibitor may be selected from the group consisting of avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B. In some embodiments, the GLP-1 RA may be selected from the group consisting of semaglutide, liraglutide, and tirzepatide, and the ACAT inhibitor may be avasimibe, CI-976, eflucimibe, or a pharmaceutically acceptable salt, derivative, or analog thereof.

Each embodiment disclosed herein is contemplated as being applicable to each of the other disclosed embodiments. Thus, all combinations of the various elements described herein are within the scope of the invention.

### BRIEF DESCRIPTION OF DRAWINGS

FIG. 1 outlines the animal study conducted according to an embodiment of the present invention.

FIG. 2 is a graph showing the effect of drug treatments on body weight change in mice fed a high-fat diet (HFD) for 18 days, named “Drug Treatments”, followed by an additional 26 days of HFD feeding, named “Drug Cessation”. “CTRL” refers to the group fed an HFD (60% fat). “SEMA0.04” refers to the group fed an HFD with daily subcutaneous injections of semaglutide (0.04 mg/kg body weight). “S0.04+A10” refers to the group fed an HFD with daily subcutaneous injections of semaglutide (0.04 mg/kg body weight) and EC-001 (10 mg/kg/body weight). “S0.02+A10” refers to the group fed an HFD with daily subcutaneous injections of semaglutide (0.02 mg/kg body weight) and EC-001 (10 mg/kg/body weight). n=9-10 mice/group. Data are expressed as mean±SEM.

FIG. 3 is a graph showing the changes in body weight (BW) of mice in the CTRL, SEMA0.04, S0.04+A10, and S0.02+A10 groups during drug treatments (i.e., Day 0-Day 18), and drug cessation (i.e., Day 18-Day 44). Data are expressed as mean±SEM.

FIG. 4 is a table showing the drug treatment days and the days to regain initial body weight after drug treatments for the mice in the SEMA0.04, S0.04+A10, and S0.02+A10 groups.

FIG. 5 is a graph showing the changes in fat weight and fat percentage of BW of mice in the CTRL, SEMA0.04, S0.04+A10, and S0.02+A10 groups, assessed by Echo-MRI at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 6. is a graph showing the changes in lean weight and lean percentage of BW of mice in the CTRL, SEMA0.04, S0.04+A10, and S0.02+A10 groups, assessed by Echo-MRI at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 7 is a graph showing the weight of individual white adipose tissue (WAT) (Epi: epididymal WAT, Ing: inguinal WAT, and Ret: retroperitoneal WAT) in mice from the CTRL, SEMA0.04, S0.04+A10 and S0.02+A10 groups, measured at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 8 is a graph showing the percentage of BW of individual white adipose tissue (WAT) (Epi: epididymal WAT, Ing: inguinal WAT, and Ret: retroperitoneal WAT) in mice from the CTRL, SEMA0.04, S0.04+A10 and S0.02+A10 groups, measured at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 9 is a graph showing the weight and percentage of BW of soleus muscle in mice from the CTRL, SEMA0.04, S0.04+A10 and S0.02+A10 groups, measured at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 10 is a graph showing the weight and percentage of BW of gastrocnemius muscle in mice from the CTRL, SEMA0.04, S0.04+A10 and S0.02+A10 groups, measured at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 11 is a graph showing the plasma glucose levels in mice in the CTRL, SEMA0.04, S0.04+A10 and S0.02+A10 groups, measured at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 12 is a graph showing the plasma total cholesterol levels in mice in the CTRL, SEMA0.04, S0.04+A10 and S0.02+A10 groups, measured at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 13 is a graph showing the plasma alanine transaminase (ALT) activity in mice in the CTRL, SEMA0.04, S0.04+A10 and S0.02+A10 groups, measured at Day 18 (During Treatments) and Day 44 (During Cessation). Data are expressed as mean±SEM. Different letters indicate significant differences at p≤0.05 as determined by One-Way ANOVA followed by Tukey's honestly significant difference test.

FIG. 14A is a graph showing the distribution of adipocyte sizes in mice from the CTRL, SEMA0.04, S0.04+A10, and S0.02+A10 groups, measured at Day 18 (During Treatments). Data are expressed as proportion (%).

FIG. 14B is a graph showing the distribution of adipocyte sizes in mice from the CTRL, SEMA0.04, S0.04+A10, and S0.02+A10 groups, measured at Day 44 (During Cessation). Data are expressed as proportion (%).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

As used herein, the term “GLP-1 RA” means a glucagon-like peptide 1 receptor agonist. The term “GLP-1 RA” refers to any agent, whether a small molecule or a large molecule, that functions as a glucagon-like peptide 1 receptor agonist, including both mono-agonists and multi-agonists. This term encompasses small molecules, including compounds, derivatives, salts, stereoisomers, hydrates, prodrugs, crystalline forms, and any other variants that retain GLP-1 receptor agonist activity. In the context of large molecules, it includes, but is not limited to, peptides, proteins, antibodies, and other biologics that exhibit GLP-1 receptor agonist activity. Non-limiting examples of the GLP-1 RA include lixisenatide, liraglutide (Victoza, Saxenda), exenatide (Byetta), exenatide extended release ((Bydurcon), albiglutide (Tanzeum), semaglutide (Ozempic, Wegovy, Rybelsus), ITCA 650, dulaglutide (Trulicity), tirzepatide (Mounjaro), retatrutide, orforglipron, lotiglipron, efpeglenatide, taspoglutide, cotadutide (MEDI0382), SAR425899, LY3298176, LY3437943, danuglipron (PF-06882961), HM15136, PF-07081532, LY3502970, BMS-986259, LY3305677, GSK2998728A, and other GLP-1 receptor agonists, dual agonists, and triple agonists under development or yet to be discovered. Accordingly, as used herein, the scope of “GLP-1 RA” expressly includes any therapeutic agent whose mechanism of action involves activation of the GLP-1 receptor, regardless of whether the activity is selective, dual, triple, or multi-target in nature. Accordingly, the scope of “GLP-1 RA” covers any derivative or structural variant that maintains GLP-1 receptor agonist activity, regardless of chemical class or molecular size.

As used herein, the term “ACAT inhibitor” refers to any small or large molecule that is capable of inhibiting the activity of acyl-coenzyme A acyltransferase. This term encompasses both natural and synthetic inhibitors, including small molecules, derivatives, analogs, salts, stereoisomers, hydrates, solvates, prodrugs, crystalline forms, and other structural variants that maintain ACAT inhibitory activity. Non-limiting examples of the ACAT inhibitor include avasimibe (CI-1011), K-604, CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511) and its analogs (analogs 1, 2, 2c and 3 or F26) (US2006/0135785), E5324, FR145237, CL277,082, YM-17E, FR129169, diethyl pyrocarbonate (Cho, et al. 2003, Biochem. Biophys. Res. Comm. 309:864-872), beauveriolides I and III (Oshiro, et al. 2007, J. Antibiotics 60:43-51), analogs of beauveriolides (258, 274, 280, 285 and 301) (Tomoda & Doi, 2008, Accounts Chem. Res. 41:32-39), Compound 1A and its derivatives (1B, 1C and 1D) (Lada, et al. 2004, J. Lipid Res. 45:378-386), methanol extracts of Saururus chinensis root containing saucerneol B and manassantin B (Lec, et al. 2004, Bioorg. Med. Chem. Lett. 14:3109-3112), and derivatives of anilidic, ureidic or diphenyl imidazole compounds (PCT/US2014/054917). Additionally, large molecules that modulate ACAT function are also included within the scope of this definition. Examples of the large molecules include peptides that specifically inhibit ACAT activity, monoclonal antibodies that target ACAT or its associated pathways, fusion proteins, and any other biologically derived entities or engineered proteins capable of inhibiting ACAT. Accordingly, the scope of the term “ACAT inhibitor” is not limited to any specific chemical structure or class, nor to the exemplary compounds disclosed herein. Rather, it broadly includes any agent-natural, synthetic, small molecule, or biologic—that demonstrates the ability to inhibit ACAT activity, whether directly or indirectly. Accordingly, the scope of “ACAT inhibitor” expressly covers derivatives, analogs, and structural variants of any ACAT inhibitory agent, provided they retain ACAT inhibitory activity.

In the scientific literature, ACAT is also known as “sterol O-acyltransferase” (SOAT), and the two terms are used interchangeably herein. The enzyme exists in at least two isoforms, ACAT1 (SOAT1) and ACAT2 (SOAT2), both of which are encompassed by the present invention. Accordingly, the term “ACAT inhibitor” as used herein refers to any agent capable of inhibiting the enzymatic activity of ACAT, whether ACAT1 (SOAT1), ACAT2 (SOAT2), or both.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes and the like.

As used herein, the term “administration” or “administering” of the subject compound refers to providing a compound of the invention and/or a prodrug thereof to a subject in need of treatment.

As used herein, the term “effective amount” or “therapeutically effective amount” refer to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose-escalation study. By way of example only, a therapeutically effective amount of a compound of the invention may be in the range of e.g., about 0.01 mg/kg/day to about 1000 mg/kg/day, from about 0.1 mg/kg/day to about 500 mg/kg/day, from about 0.1 mg (×2)/kg/day to about 500 mg (×2)/kg/day.

In addition, such compounds and compositions may be administered singly or in combination with one or more additional therapeutic agents. The methods of administration of such compounds and compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration, optic administration, or transdermal administration. Compounds provided herein may be administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such pharmaceutical compositions are formulated as tablets, pills, capsules, a liquid, an inhalant, a nasal spray solution, a suppository, a solution, a gel, an emulsion, an ointment, eye drops, car drops, or microneedle patches.

The therapeutically effective amount may vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

The compounds described herein include all stereoisomers, geometric isomers, tautomers, isotopes, and prodrug of the structures depicted. The compounds described herein can be present in various forms including crystalline, powder and amorphous forms of those compounds, pharmaceutically acceptable salts, including, for example, polymorphs, pseudopolymorphs, solvates, hydrates, unsolvated polymorphs (including anhydrates), conformational polymorphs, and amorphous forms of the compounds, as well as mixtures thereof.

As used herein, the term “pharmaceutically acceptable” material refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

As used herein, the term “pharmaceutically acceptable salt” refers to a formulation of a compound that does not cause significant irritation to an organism to which it is administered and does not abrogate the biological activity and properties of the compounds described herein.

Pharmaceutically acceptable salt forms may include pharmaceutically acceptable acidic/anionic or basic/cationic salts (UK Journal of Pharmaceutical and Biosciences Vol. 2 (4), 01-04, 2014, which is incorporated herein by reference). Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and tricthiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine, and triethanolamine salts.

A pharmaceutically acceptable acid addition salt of a compound of the invention may be prepared by methods known in the art and may be formed by reaction of the free base form of the compound with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, and hexanoic acid. A pharmaceutically acceptable acid addition salt can comprise or be, for example, a hydrobromide, hydrochloride, sulfate, bisulfate, nitrate, phosphate, succinate, maleate, formarate, acetate, propionate, fumarate, citrate, tartrate, lactate, benzoate, carbonate, benzathine, chloroprocaine, choline, histidine, meglumine, meglumine, procaine, triethylamine, besylate, decanoate, ethylenediamine, salicylate, glutamate, aspartate, p-toluenesulfonate, benzenesulfonate, methanesulfonate, ethanesulfonate, naphthalenesulfonate (e.g., 2-naphthalenesulfonate), and hexanoate salt.

A pharmaceutically acceptable base addition salt of a compound of the invention may also be prepared by methods known in the art and may be formed by the reaction of the free base form of the compound with a suitable inorganic or organic base including, but not limited to, hydroxide or other salt of sodium, potassium, lithium, ammonium, calcium, magnesium, iron, zinc, copper, manganese, aluminum, tromethamine, glycolate, hydrabamine, methylbromide, methylnitrate, octanoate, oleate, and the like.

A free acid or free base form of a compound of the invention may be prepared by methods known in the art (e.g., for further details see L. D. Bigley, S. M. Berg, D. C. Monkhouse, in “Encyclopedia of Pharmaceutical Technology”. Eds, J. Swarbrick and J. C. Boylam, Vol 13, Marcel Dekker, Inc., 1995, pp. 453-499, which is incorporated herein by reference). For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Aspects of this disclosure include prodrug forms of any of the compounds described herein. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)).

Prodrug derivatives of the compounds of the invention may be prepared by methods known to those of ordinary skill in the art (e.g., for further details see Saulnier et al., Bioorg. Med. Chem. Letters, 1994, 4, 1985, which is incorporated herein by reference). Protected derivatives of the compounds of the invention may be prepared by means known to those of ordinary skill in the art. A detailed description of techniques applicable to the creation of protecting groups and their removal can be found in T. W. Greene, “Protecting Groups in Organic Chemistry,” 3rd edition, John Wiley and Sons, Inc., 1999 and “Design of Prodrugs”, ed. 11. Bundgaard, Elsevier, 1985, which are incorporated herein by reference.

The compounds of the present disclosure may be prepared as stereoisomers. Where the compounds have at least one chiral center, they may exist as enantiomers. Where the compounds possess two or more chiral centers, they may exist as diastereomers. The compounds of the invention may be prepared as racemic mixtures. Alternatively, the compounds of the invention may be prepared as their individual enantiomers or diastereomers by reaction of a racemic mixture of the compound with an optically active resolving agent to form a pair of diastereo-isomeric compounds, separating the diastereomers, and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet and Samuel H. Wilen, “Enantiomers, Racemates and Resolutions” John Wiley And Sons, Inc., 1981, which is incorporated herein by reference.

The compounds of the invention may be prepared as solvates (e.g., hydrates). The term “solvate” refers to a complex of variable stoichiometry formed by a solute (for example, a compound of the invention or a pharmaceutically acceptable salt thereof) and a solvent. Such solvents for the purpose of the invention may not interfere with the biological activity of the solute. Non-limiting examples of suitable solvents include water, acetone, methanol, ethanol and acetic acid. Preferably the solvent used is a pharmaceutically acceptable solvent.

Furthermore, the compounds of the invention may be prepared as crystalline forms. The crystalline forms may exist as polymorphs.

It should be noted that in view of the close relationship between the compound of the invention and their other forms, whenever a compound is referred to in this context herein, a corresponding salt, diastereomer, enantiomer, racemate, crystalline, polymorph, prodrug, hydrate, or solvate is also intended, if it is possible or appropriate under certain circumstances.

Another aspect of the present invention provides a composition for controlling body weight. The composition comprises a therapeutically effective amount of one or more ACAT inhibitors and a therapeutically effective amount of one or more GLP-1 RAs.

As used herein, the term “composition” is intended to encompass a product comprising the compound, salt, diastereomer, enantiomer, racemate, hydrate, solvate, or a pharmaceutical combination thereof in the therapeutically effective amount, as well as any other product which results, directly or indirectly, from claimed compound, salt, diastereomer, enantiomer, racemate, hydrate, solvate, or a pharmaceutical combination thereof.

As used herein, the term “pharmaceutical composition” refers to a mixture of a therapeutically active component (ingredient) with one or more other components, which may be chemically or biologically active or inactive. Such components may include, but not limited to, carriers, stabilizers, diluents, dispersing agents, suspending agents, thickening agents, excipients, and adjuvants.

As used herein, the term “pharmaceutical combination” means a product that results from the mixing or combining of more than one therapeutically active ingredient.

As used herein, the term “acceptable” with respect to a formulation, composition or ingredient, as used herein, means having no persistent detrimental effect on the general health of the subject being treated.

As used herein, the term “carrier” refers to chemical or biological material that can facilitate the incorporation of a therapeutically active ingredient(s) into cells or tissues.

Suitable excipients may include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone), and lubricants (e.g., magnesium stearate, talc, stearic acid and sodium lauryl sulphate).

Any suitable pharmaceutically acceptable carriers, stabilizers, diluents, dispersing agents, suspending agents, thickening agents, excipients, and adjuvants known to those of ordinary skill in the art for use in pharmaceutical compositions may be selected and employed in the compositions described herein. The compositions described herein may be in the form of a solid, liquid, or gas (aerosol). For example, they may be in the form of tablets (coated tablets) made of, for example, collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another), and aerosols (propellant-containing or -free inhale solutions). Also, the compositions described herein may be formulated for sustained or slow release.

Other embodiments and uses will be apparent to one skilled in the art in light of the present disclosures. The following examples are provided merely as illustrative of various embodiments and shall not be construed to limit the invention in any way.

### EXAMPLES

### Example 1: Materials and Methods

### 1. Animal Husbandry and Administration of Drugs

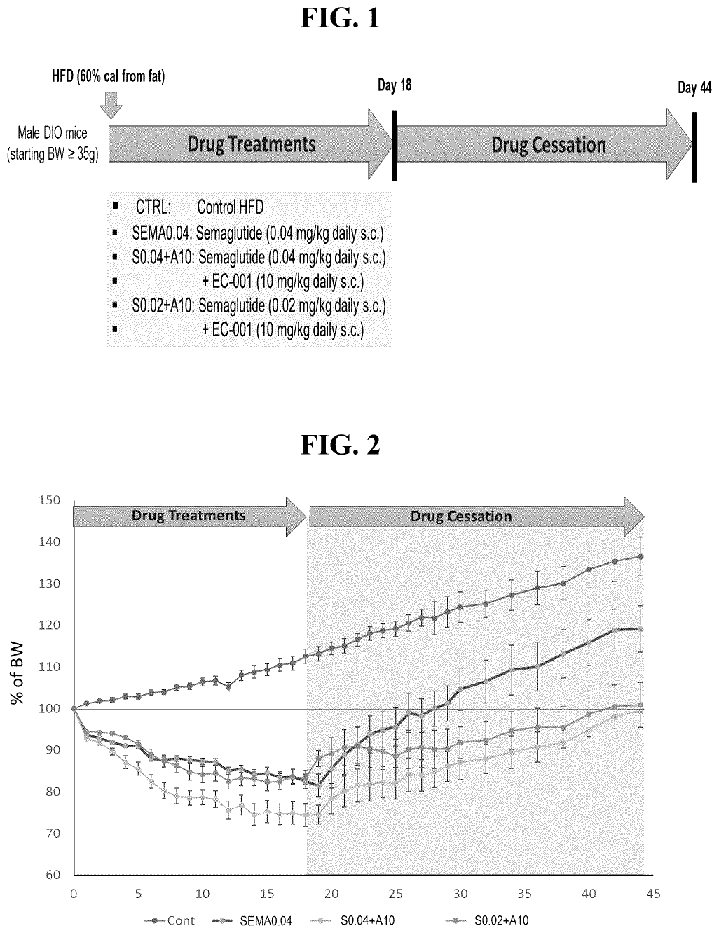

Six- to eight-week-old high-fat diet-induced obese (DIO) male C57BL/6 mice were purchased from the Jackson Laboratory (Bar Harbor, ME, USA). These mice were then randomly assigned to CTRL, SEMA0.04, S0.04+A10 and S0.02+A10 groups and fed with HFD. EC-001 solution was prepared for making the final concentration of EC-001 at 10 mg/kg BW, as described in Y. Zhu, S. Q. Kim, Y. Zhang, Q. Liu, K. H. Kim, Pharmacological inhibition of acyl-coenzyme A: cholesterol acyltransferase alleviates obesity and insulin resistance in diet-induced obese mice by regulating food intake, Metabolism 123 (2021) 154861, which is incorporated herein by reference. Semaglutide stock solution prepared in DMSO (1 mg/ml) was diluted with PBS for the final concentration of 0.04 mg/kg BW. The drug solution or the vehicle solution at a volume of 10 μl/g BW was injected to the HFD fed mice sc daily from Day 0 to Day 18. After Day 18, mice were fed with HFD with no drug treatments until Day 44 (FIG. 1) Mice were kept on a 12/12 h light/dark cycle in a humidity and temperature control facility with ad libitum access to food and water.

### 2. Body Composition and Blood Analysis

Body composition (e.g., fat mass, lean mass, free water, and total water) changes were assessed right before sacrificing the second cohort of mice using EchoMRI (Echo Medical Systems). At the end of the feeding period, mice were starved for 8 hr and anesthetized carbon dioxide. Blood samples were collected in tubes containing 3.5 mg EDTA by cardiac puncture, frozen and stored until analysis. Adipose tissue, liver and muscle (e.g., soleus and gastrocnemius muscles) were snap-frozen in liquid nitrogen and stored at −80° C. Plasma lipids were determined enzymatically using a Cholesterol Reagent and triglyceride (TG) assay kits for total cholesterol (TC) and TG measurements, respectively. Plasma alanine transaminase (ALT) activity was measured using an assay kit. A Liquid Glucose (Oxidase) reagent set was used to determine plasma glucose concentrations according to the manufacturer's protocol.

### Statistical Analysis

Data were presented as mean±SEM. Statistical analyses were performed with GraphPad Prism 5 program (GraphPad Software, Inc.). Two-tailed Student t-tests or One-Way ANOVA followed by the Tukey's pos-hoc test were performed to analyze the statistical significance of the results. P<0.05 was considered statistically significant.

### Example 2: Alleviation of Body Weight Regain after GLP-1RA and EC-001 Combined Treatment in DIO Mice

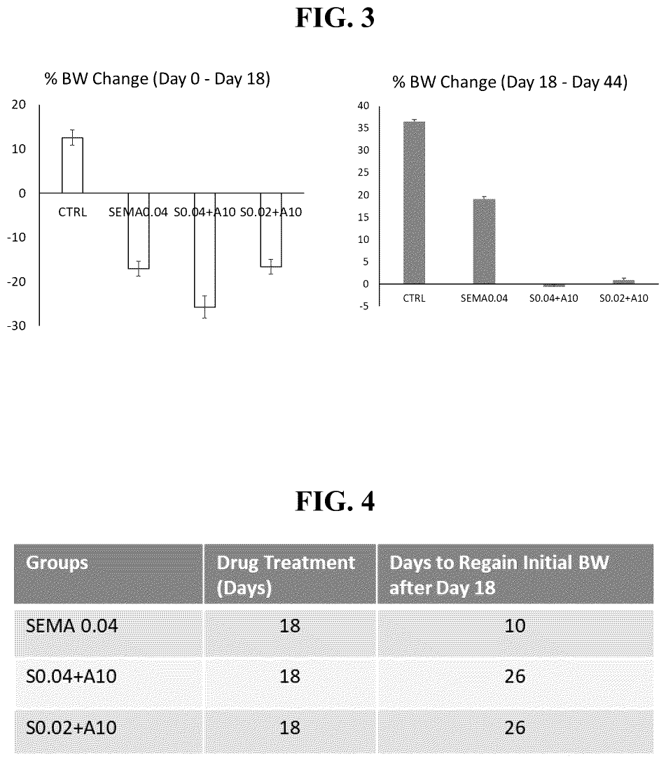

Throughout the initial 18 days of drug treatments, the mean body weight of the CTRL group increased by 12.6% compared to their starting body weight. In contrast, the mean body weight changes for mice in the SEMA0.04 monotherapy group, the S0.04+A10 and S0.02+A10 combination groups were −17.1%, −25.8%, and −16.6% of their starting body weight, respectively (FIGS. 2 and 3). These results indicate that significant reductions in body weight were observed in all treatment groups compared to the control. Specifically, relative to the CTRL group, the SEMA0.04 monotherapy group showed a 29.7% greater reduction in body weight, the S0.04+A10 combination group showed a 38.4% greater reduction, and the S0.02+A10 combination group showed a 29.2% greater reduction. Moreover, compared to the SEMA0.04 monotherapy group, the S0.04+A10 combination group achieved an additional 50.9% greater reduction in body weight, and the S0.02+A10 combination group demonstrated a reduction that was essentially comparable. In an unforseen way, these findings demonstrate that the combination of a GLP-1 receptor agonist (GLP-1 RA) and an ACAT inhibitor produces a synergistic and more pronounced reduction in body weight than GLP-1 RA monotherapy. Importantly, such combination therapy enables a substantial lowering of the effective dose of the GLP-1 RA while still maintaining comparable, if not superior, efficacy.

After an additional 26 days following the cessation of drug treatments, the mean body weight of the CTRL group increased by 36.5% compared to their weight at Day 18. The mean body weight changes for the mice in the SEMA0.04 monotherapy group, the S0.04+A10 combination group, and the S0.02+A10 combination group were 19.1%, −0.7%, and −0.8% of their Day 18 body weights, respectively (FIGS. 2˜4). Notably, the SEMA0.04 monotherapy group exhibited a clear rebound in body weight, and the rate of regain, as reflected by the slope of the curve, was even faster than that observed in the CTRL group, although starting from a lower baseline. In striking contrast, both the S0.04+A10 combination group and the S0.02+A10 combination group displayed markedly attenuated rebound kinetics, with body weight remaining essentially stable relative to Day 18 throughout the observation period. When the time to return to baseline (Day 0) body weight was compared, the SEMA0.04 monotherapy group rebounded within approximately 10 days after drug cessation, whereas the S0.04+A10 combination group and the S0.02+A10 combination group required about 26 and 24 days, respectively. These findings indicate that only the combination of a GLP-1 receptor agonist and an ACAT inhibitor provided durable suppression of post-treatment rebound, producing a delayed and flattened regain profile that was not achieved with GLP-1 RA monotherapy. Importantly, this unexpected synergistic effect suggests that the combination therapy extends therapeutic benefit beyond the dosing period and enables lowering of the effective GLP-1 RA dose while maintaining efficacy.

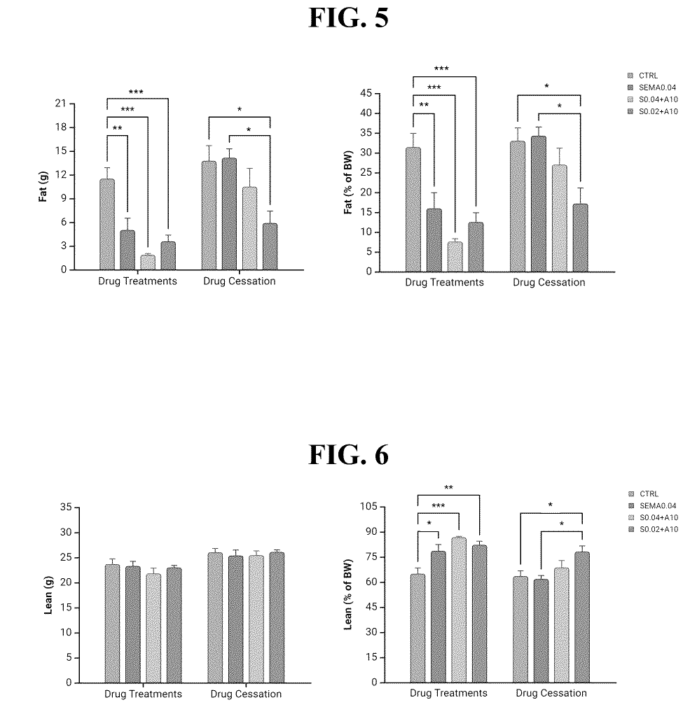

During the drug treatment period, the mice in the SEMA0.04 group displayed a 56.3% reduction in fat mass and a 49.1% lower percentage of body weight derived from fat compared to the CTRL group (FIG. 5). The S0.04+A10 group showed an even greater effect, with an 83.5% reduction in fat mass and a 75.8% lower percentage of body weight relative to the CTRL group, corresponding to a 1.53-fold greater fat reduction index compared to monotherapy. The S0.02+A10 group also demonstrated marked suppression, showing a 68.7% decrease in fat mass and a 60.0% lower percentage of body weight compared to the CTRL group, with a 1.29-fold improvement over monotherapy. These results confirm that all treatment groups significantly reduced fat accumulation, with the combination of a GLP-1 receptor agonist and an ACAT inhibitor producing the most pronounced and unexpected reductions beyond those achieved by GLP-1 RA monotherapy.

During the drug cessation period, the mice in the S0.02+A10 group maintained a durable effect, displaying 57.1% less fat mass and 47.9% lower percentage of body weight relative to the CTRL group (FIG. 5). Importantly, when quantified by relative rebound suppression indices, the S0.04+A10 and S0.02+A10 groups exhibited 1.49-fold and 1.71-fold greater suppression of fat rebound, respectively, compared to GLP-1 RA monotherapy. Although statistical significance was not reached among the CTRL, SEMA0.04, and S0.04+A10 groups at this stage, the numerical values for both combination groups remained consistently lower than those of the monotherapy and control groups. Remarkably, this trend demonstrates a synergistic effect whereby the combination therapy not only enhances fat reduction during treatment but also provides sustained suppression of fat rebound following drug withdrawal—an effect that was not achieved with GLP-1 RA monotherapy.

Absolute lean mass did not differ significantly among the groups during either the treatment or cessation periods, indicating that neither GLP-1 RA monotherapy nor its combination with an ACAT inhibitor caused measurable loss of lean tissue (FIG. 6). However, when expressed as a percentage of body weight, the combination groups consistently outperformed the GLP-1 RA monotherapy group.

During the treatment period, both the S0.04+A10 combination group and the S0.02+A10 combination group exhibited significantly higher lean percentages compared to the GLP-1 RA monotherapy group, demonstrating that the weight reduction achieved by the combination therapy was primarily attributable to fat loss rather than lean tissue depletion. Quantitatively, the relative index for lean preservation was 1.14 in S0.04+A10 and 1.08 in S0.02+A10 compared to monotherapy, underscoring the superior capacity of the combination to maintain lean mass.

During the drug cessation period, this advantage was surprisingly maintained: both combination groups continued to show higher lean percentages than the GLP-1 RA monotherapy group, despite the overall rebound in body weight. Importantly, the relative indices for lean recovery were 1.10 in S0.04+A10 and 1.07 in S0.02+A10, further confirming that the combination groups preserved muscle composition more effectively during rebound. In particular, the S0.02+A10 group displayed 57.1% less fat mass and 47.9% lower fat-derived body weight percentage relative to the CTRL group, while simultaneously maintaining a higher lean percentage compared to the GLP-1 RA monotherapy group.

Notably, these findings demonstrate that the combination of a GLP-1 receptor agonist and an ACAT inhibitor not only synergistically enhances fat reduction but also preserves and enriches lean composition, thereby improving the quality of weight loss. This synergistic effect provides a clear therapeutic advantage over GLP-1 RA monotherapy by extending efficacy beyond simple weight reduction and addressing the critical clinical challenge of muscle preservation during obesity treatment.

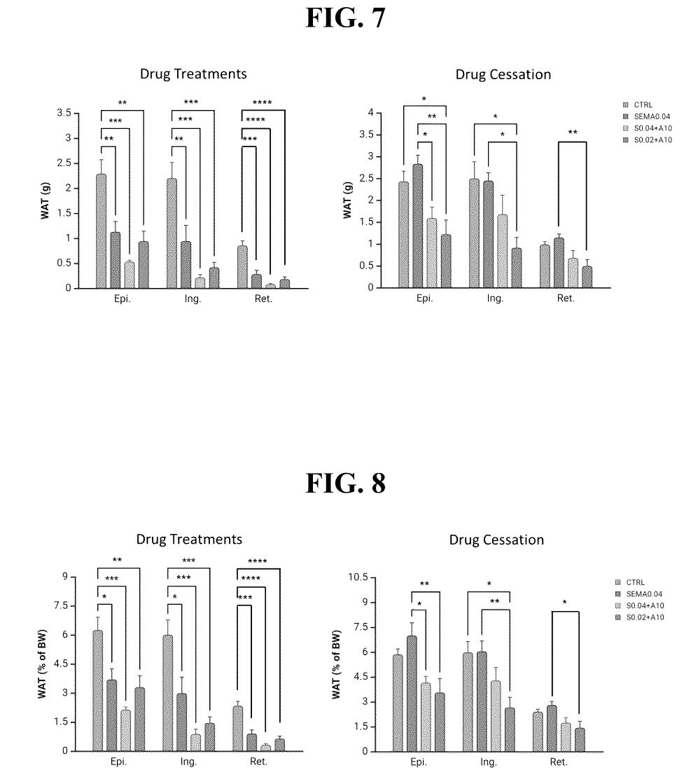

During the drug treatment period, the mice in the SEMA0.04, S0.04+A10, and S0.02+A10 groups exhibited significantly lower epididymal, inguinal, and retroperitoneal white adipose tissue (WAT) mass compared to the CTRL group (FIG. 7). The reductions were most pronounced in the combination groups, confirming that the fat-lowering effect of treatment was not confined to total body fat but extended consistently across multiple visceral and subcutaneous depots.

Surprisingly, after discontinuation of drug treatments, the S0.02+A10 group maintained significantly lower epididymal, inguinal, and retroperitoneal WAT mass compared to both the CTRL and SEMA0.04 groups, while the S0.04+A10 group also demonstrated a trend toward lower depot weights relative to GLP-1 RA monotherapy (FIG. 7). These results demonstrate that the combination of a GLP-1 receptor agonist and an ACAT inhibitor produces a synergistic and more durable suppression of visceral and subcutaneous adipose depots than GLP-1 RA monotherapy alone. Importantly, this unexpected synergistic effect indicates that such combination therapy not only augments fat reduction during treatment but also sustains depot-specific suppression of WAT rebound after drug withdrawal, thereby providing a distinct therapeutic advantage over GLP-1 RA monotherapy.

Similarly, during the drug treatment period, the mice in the SEMA0.04, S0.04+A10, and S0.02+A10 groups exhibited significantly lower white adipose tissue (WAT) percentage of body weight compared to the CTRL group (FIG. 8). The reductions were particularly pronounced in the combination groups, indicating that the decrease in body fat proportion was not only a function of absolute fat mass but also reflected a favorable shift in body composition.

Contrary to expectations, after discontinuation of drug treatments, the S0.02+A10 group maintained significantly lower WAT percentage of body weight compared to both the CTRL and SEMA0.04 groups (FIG. 8). This finding demonstrates that the combination of a GLP-1 receptor agonist and an ACAT inhibitor provides a synergistic and durable suppression of body fat proportion that extends beyond the treatment period. Importantly, such an effect was not achieved by GLP-1 RA monotherapy, thereby underscoring the therapeutic advantage of combination therapy in both enhancing fat reduction and sustaining improved body composition after treatment withdrawal.

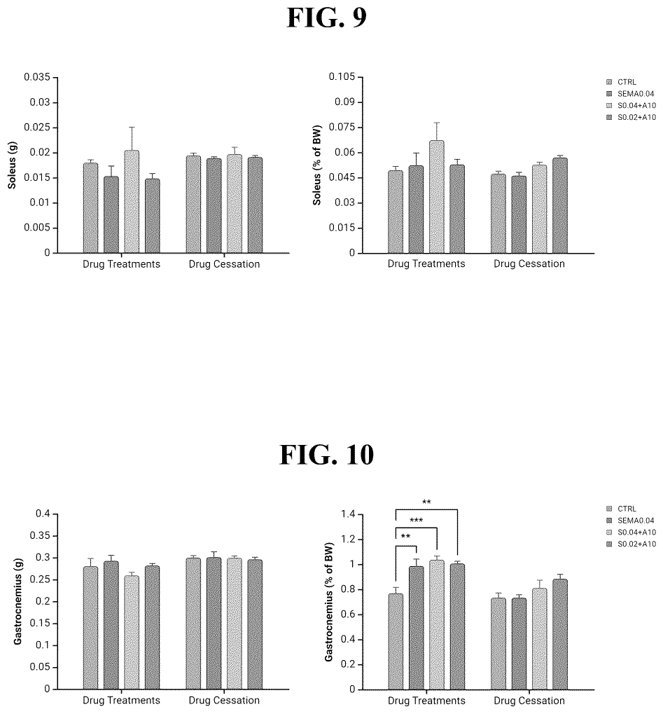

There were no significant differences in soleus muscle weight or percentage of body weight among the four groups during either the treatment or cessation periods, indicating that neither GLP-1 RA monotherapy nor its combination with an ACAT inhibitor caused measurable loss of soleus muscle mass (FIG. 9). Similarly, no significant differences were observed in gastrocnemius muscle weight among the groups (FIG. 10). These findings demonstrate that administration of these agents, either alone or in combination, did not adversely affect skeletal muscle preservation.

Interestingly, the gastrocnemius muscle percentage of body weight was significantly higher in the SEMA0.04, S0.04+A10, and S0.02+A10 groups compared to the CTRL group during the treatment period. Moreover, although statistical significance was not reached, both combination groups exhibited numerically higher gastrocnemius muscle percentages than the SEMA0.04 monotherapy group, suggesting a trend toward superior muscle preservation when GLP-1 RA was combined with an ACAT inhibitor. Collectively, these results demonstrate that the combination of a GLP-1 receptor agonist and an ACAT inhibitor not only synergistically enhances fat reduction but also preserves and relatively enriches skeletal muscle composition. This unexpected synergistic effect improves the quality of weight reduction by maintaining muscle mass while lowering fat mass, thereby providing a therapeutic advantage over GLP-1 RA monotherapy.

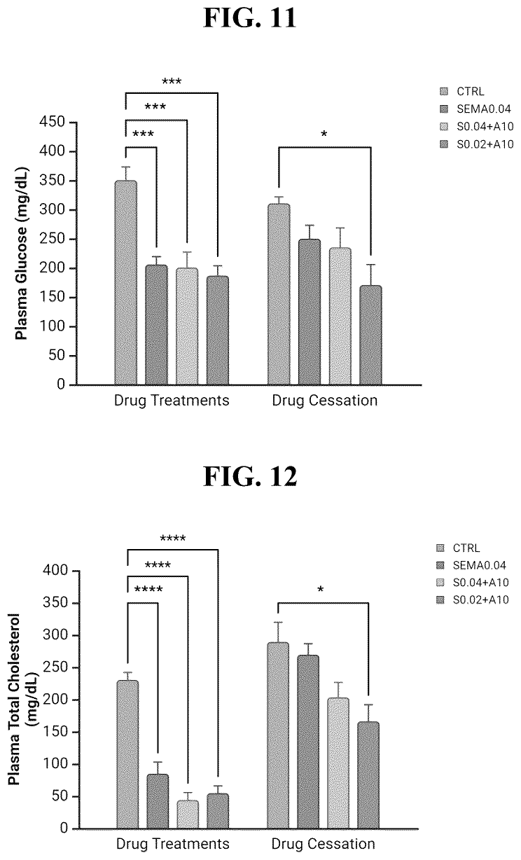

During the treatment period (Day 0-Day 18), the combined drug treatments resulted in a marked reduction in plasma glucose levels compared to the CTRL group (FIG. 11). In addition, relative to the GLP-1 RA monotherapy group, the S0.04+A10 and S0.02+A10 groups achieved 3% and 8% greater glucose reduction, respectively, as reflected by the relative glucose reduction indices of 1.03 and 1.08 compared to 1.00 for monotherapy.

After discontinuation of drug treatments (Day 18-Day 44), plasma glucose levels rebounded in all groups; however, the rebound was markedly attenuated in the combination groups. Specifically, the S0.04+A10 and S0.02+A10 groups exhibited 30% and 150% stronger suppression of glucose rebound, respectively, compared to GLP-1 RA monotherapy, as indicated by rebound suppression indices of 1.30 and 2.50 versus 1.00 for monotherapy.

Counterintuitively, these findings demonstrate that the combination of a GLP-1 receptor agonist and an ACAT inhibitor not only enhances the acute glucose-lowering efficacy during active treatment but also provides durable protection against post-treatment rebound. Such synergistic effects are of particular clinical importance, as they suggest that combination therapy may substantially reduce cardiovascular risk associated with GLP-1 RA-based treatments for diabetes and obesity.

During the treatment period (Day 0-Day 18), the combined drug treatments produced a profound reduction in plasma total cholesterol levels compared to the CTRL group (FIG. 12). Also, relative to the GLP-1 RA monotherapy group, the S0.04+A10 and S0.02+A10 groups achieved 80% and 50% greater cholesterol reduction, respectively, as reflected by the relative cholesterol reduction indices of 1.80 and 1.50 compared to 1.00 for monotherapy.

After discontinuation of drug treatments (Day 18-Day 44), plasma cholesterol levels rebounded in all groups; however, the rebound was strongly suppressed in the combination groups. Specifically, the S0.04+A10 and S0.02+A10 groups demonstrated 12% and 47% greater suppression of cholesterol rebound, respectively, compared to GLP-1 RA monotherapy, as indicated by rebound suppression indices of 1.12 and 1.47 versus 1.00 for monotherapy.

Remarkably, these results demonstrate that the combination of a GLP-1 receptor agonist and an ACAT inhibitor not only potentiates cholesterol-lowering efficacy during active treatment but also provides sustained suppression of rebound after treatment withdrawal. Such a synergistic effect suggests that combination therapy may substantially reduce cardiovascular risk factors-including hyperglycemia and hypercholesterolemia—that frequently accompany GLP-1 RA-based therapies for diabetes and obesity.

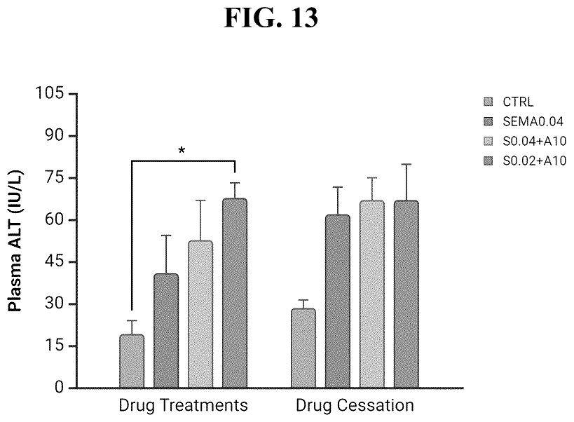

To evaluate potential liver toxicity associated with the combination treatment, plasma alanine aminotransferase (ALT) levels were measured (FIG. 13). During the treatment period, ALT levels in the S0.02+A10 group were elevated relative to the CTRL group. However, the ALT levels in the S0.04+A10 group were not significantly different from either the CTRL group or the SEMA0.04 monotherapy group. Moreover, the S0.02+A10 group did not differ statistically from either the SEMA0.04 or S0.04+A10 groups.

Importantly, after treatment cessation, no significant differences in ALT levels were observed among any of the groups, indicating that the observed elevation was transient and fully reversible. Furthermore, although not shown, histological examination of liver tissue revealed no evidence of hepatic damage in any group.

Collectively, these findings demonstrate that combination therapy with a GLP-1 receptor agonist and an ACAT inhibitor does not induce persistent or clinically meaningful hepatotoxicity. Rather, both plasma ALT measurements and histological analyses support a favorable hepatic safety margin for the combination treatment.

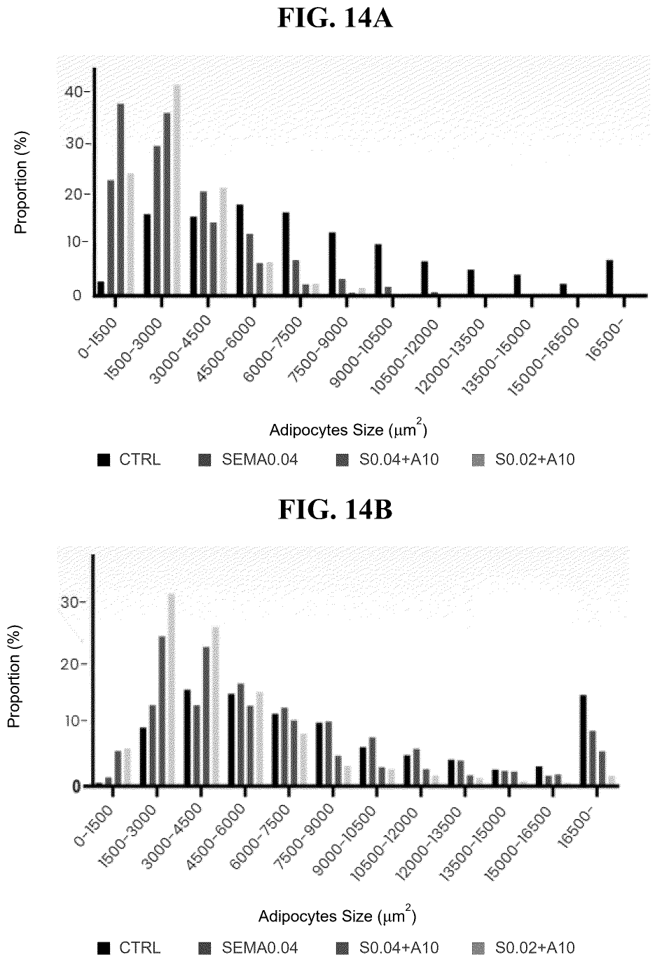

In addition, adipocyte size distribution was analyzed to evaluate the qualitative changes in white adipose tissue (WAT) morphology under each treatment condition (FIGS. 14A and 14B). During the treatment period (FIG. 14A), both combination groups (S0.04+A10 and S0.02+A10) exhibited a pronounced reduction in the proportion of large adipocytes (≥6,000 μm²) and an increase in the proportion of small adipocytes (≤3,000 μm²) compared with the GLP-1 RA monotherapy group. Quantitatively, relative to monotherapy, the S0.04+A10 combination group reduced large adipocytes by 1.23-fold and increased small adipocytes by 1.69-fold, while the S0.02+A10 combination group reduced large adipocytes by 1.37-fold and increased small adipocytes by 2.00-fold. These findings demonstrate that the combination of a GLP-1 RA and an ACAT inhibitor not only reduces fat mass but also remodels adipose tissue toward a healthier profile characterized by smaller adipocytes.

After drug withdrawal (FIG. 14B), this remodeling effect persisted. Both combination groups maintained a lower proportion of large adipocytes and a higher proportion of small adipocytes relative to monotherapy. Specifically, the S0.04+A10 combination group suppressed rebound of large adipocytes by 1.60-fold and preserved small adipocytes by 1.33-fold, while the S0.02+A10 combination group achieved 2.00-fold and 1.33-fold effects, respectively. Importantly, this sustained shift in adipocyte size distribution is likely to contribute to the attenuation of body weight rebound observed in the combination groups, thereby providing a mechanistic basis for the durable anti-obesity benefits beyond the dosing period.

Those skilled in the art will recognize that numerous modifications can be made to the specific implementations described above. The implementations should not be limited to the particular limitations described. Other implementations may be possible.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A method for managing body weight and metabolic parameters in a subject in need thereof, the method comprising administering to the subject a therapeutically effective amount of a combination of one or more GLP-1 receptor agonists (GLP-1 RAs) and one or more ACAT inhibitors, wherein the combination therapy is effective to achieve one or more of the following:
a. delaying and reducing weight regain after cessation of the treatment compared to GLP-1 RA monotherapy;
b. minimizing or suppressing the rebound in blood cholesterol levels after cessation of the treatment compared to GLP-1 RA monotherapy; and
c. minimizing or suppressing the rebound in blood glucose levels after cessation of the treatment compared to GLP-1 RA monotherapy.

2. The method according to claim 1, wherein said GLP-1 RA is selected from the group consisting of lixisenatide, liraglutide, exenatide, albiglutide, semaglutide, dulaglutide, tirzepatide, retatrutide, orforglipron, lotiglipron, efpeglenatide, and taspoglutide.

3. The method according to claim 1, wherein said ACAT inhibitor is selected from the group consisting of avasimibe, CI-976, eflucimibe, and a pharmaceutically acceptable salt, derivative, or analog thereof.

4. The method according to claim 1, wherein said GLP-1 RA and said ACAT inhibitor are formed in a single formulation and the formulation is administered.

5. The method according to claim 1, wherein said GLP-1 RA and said ACAT inhibitor are formulated separately and administered simultaneously or sequentially.

6. The method according to claim 1, wherein said GLP-1 RA is liraglutide, semaglutide, or tirzepatide, and said ACAT inhibitor is avasimibe (CI-1011), K-604, CI-976, eflucimibe (F12511), or a pharmaceutically acceptable salt, derivative, or analog thereof.

7. The method according to claim 1, wherein the combination therapy is effective to further achieve one or more of the following:
a. enabling the use of a lower dose of GLP-1 RA compared to GLP-1 RA monotherapy; and
b. reducing or preventing muscle loss during the treatment period or the post-treatment period compared to GLP-1 RA monotherapy.

8. A pharmaceutical composition for managing body weight and metabolic parameters in a subject in need thereof, the composition comprising one or more GLP-1 receptor agonists (GLP-1 RAs) and one or more ACAT inhibitors, wherein the composition is formulated to achieve one or more of the following:
a. delaying or reducing weight regain after cessation of the treatment compared to GLP-1 RA monotherapy;
b. minimizing or suppressing the rebound in blood cholesterol levels after cessation of the treatment compared to GLP-1 RA monotherapy; and
c. minimizing or suppressing the rebound in blood glucose levels after cessation of the treatment compared to GLP-1 RA monotherapy.

9. The pharmaceutical composition according to claim 8, wherein said GLP-1 RA is selected from the group consisting of lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide.

10. The pharmaceutical composition according to claim 8, wherein said ACAT inhibitor is selected from the group consisting of avasimibe, CI-976, eflucimibe, and a pharmaceutically acceptable salt, derivative, or analog thereof.

11. The pharmaceutical composition according to claim 8, wherein said GLP-1 RA is liraglutide, semaglutide, or tirzepatide, and said ACAT inhibitor is avasimibe (CI-1011), K-604, CI-976, eflucimibe (F12511), or a pharmaceutically acceptable salt, derivative, or analog thereof.

12. The pharmaceutical composition according to claim 8, wherein the composition is formulated to achieve one or more of the following:
a. enabling the use of a lower dose of GLP-1 RA compared to GLP-1 RA monotherapy; and
b. reducing or preventing muscle loss during the treatment period or the post-treatment period compared to GLP-1 RA monotherapy.
